(19) United States
(12) Patent Application Publication
(10) Pub. No.: US 2007/0043408 A1
(43) Pub. Date: Feb. 22, 2007

(54) Temperature treatment off-loading device

(76) Inventors: Richard Dan Winnett, Memphis, TN (US); Larry Lavery, Temple, TX (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/417,012

(22) Filed: May 2, 2006

Related U.S. Application Data
(60) Provisional application No. 60/681,076, filed on May 13, 2005.

(51) Int. Cl. A61F 7/00

(52) U.S. Cl. 607/96

(57) ABSTRACT

The present invention provides for a temperature treatment off-loading device configured to relieve pressure from a target site while simultaneously lowering the temperature of the target site. The present invention is preferably utilized to treat a neuropathic foot by relieving pressure and controlling the temperature of a target site, which may be a site that may develop an ulcer or a “hot spot,” on the foot.

### CROSS-REFERENCE TO RELATED APPLICATIONS

This application claims the benefit of U.S. Provisional Application No. 60/681,076, filed May 13, 2005, the disclosure of which is expressly incorporated by reference.

### FIELD OF THE INVENTION

This invention relates to devices and methods for providing neuropathy relief, and specifically to devices and methods for providing patients with neuropathy relief from hot spots and pressure points to prevent the onset of an ulcer. The invention can be used to treat inflammatory injuries in persons with diabetes and sensory neuropathy such as Charcot's arthroplathy and to prevent neuropathic ulcerations. In addition, the invention can be used to treat lower extremity injuries such as ankle sprains, fractures, and to decrease post operative inflammation and pain after foot and ankle surgery.

### BACKGROUND

Foot ulcers are one of the most common precursors to diabetes related amputations. Pecoraro R E, Reiber G E, and Burgess E M, Causal pathways to amputation: basis for prevention, Diabetes Care., 1990, 13: p. 513-521; Pecoraro R E, Chronology and determinants of tissue repair in diabetic lower extremity ulcers, Diabetes, 1991, 40: p. 1305-1313. Other factors that have been associated with amputation, such as infection, faulty wound healing, and ischemia, usually do not cause tissue loss or amputation in the absence of a wound. Therefore, ulcer prevention is one of the foci of any amputation prevention program.

One of the most common mechanisms in the development of neuropathic foot ulcerations and neuropathic fractures involves a cumulative effect of unrecognized repetitive trauma at pressure points on the sole of the foot over the course of several days. Lavery L A, et al., Practical Criteria to Screen Patients at Risk for Diabetic Foot Ulceration, Arch Int Med, 1998, 158: p. 157-162; Reiber G E, et al., Causal Pathways for Incident Lower-Extremity Ulcers in Patients with Diabetes from to Settings, Diabetes Care, 1999, January; 22(1): p. 157-62; Frykberg R G, et al., Role of Neuropathy and High Foot Pressures in Diabetic Foot Ulceration, Diabetes Care, 1998, 21: p. 1714-9. The standard approach to prevent ulceration is to provide padded insoles and protective shoes, educate the patient and their family, and provide regular foot inspection by the patients' primary care physician or podiatrist.

Several studies have demonstrated a significant decrease in the incidence of ulceration and re-ulceration when this type of approach is used. Chantelau E and Haage P, An audit of cushioned diabetic footwear: relation to patient compliance, Diabetic Medicine, 1994, (11): p. 114-116; Edmonds M E, et al., Improved survival of the diabetic foot: The role of a specialized foot clinic, QJ med., 1986, 60: p. 763-771; Uccioli L, et al., Manufactured shoes in the prevention of diabetic foot ulcers, Diabetes Care, 1995, 18(10): p. 1376-1378. For instance, Edmonds and coworkers reported a 26% re-ulceration rate in high-risk diabetic patients with special shoes and insoles compared to an 83% re-ulceration rate in patients that wore their own shoes. Edmonds M E, et al., Improved survival of the diabetic foot: The role of a specialized foot clinic, QJ med., 1986, 60: p. 763-771. Likewise Uccioli reported a 28% re-ulceration rate in diabetic patients treated with custom shoes and insoles compared to 58% re-ulceration among patients without special footwear. Uccioli L, et al., Manufactured shoes in the prevention of diabetic foot ulcers, Diabetes Care, 1995, 18(10): p. 1376-1378. All of these studies were performed at specialty foot centers with a focus on diabetic foot care. Even in these centers, however, the incidence of re-injury was still very high.

Except for traumatic wounds, areas that are likely to ulcerate have been associated with increased local skin temperatures due to inflammation and enzymatic autolysis of tissue. LAVERY L A, Higgins K R, Lanctot D R, Constantinides G P, Zamorano R G. Armstrong G, Athanasiou K A, Agrawal C M Home monitoring of foot skin temperatures to prevent ulceration Diabetes Care. 2004 November; 27(11):2642- Armstrong D G, et al., Infrared dermal thermometry of the high risk diabetic foot, Physical Therapy, 1997, 77: p. 169-177; Clark R P, et al., Thermography and pedobarography in the assessment of tissue damage in neuropathic and atherosclerotic feet, Thermology, 1988, 3: p. 15-20; Brand P W, The insensitive foot (including leprosy), in Disorders of the Foot and Ankle, Jahss M, Editor, 1991, Saunders: Philadelphia. p. 2173-2175. Identifying areas of injury by the presence of inflammation would then allow patients or health care providers to take action to decrease the inflammation before a wound develops. Our rationale for evaluating skin temperatures involves the search for a quantifiable measurement of inflammation that can be used to identify pathologic processes before they result in ulcers. Inflammation is one of the earliest signs of foot ulceration. It is characterized by five cardinal signs: redness, pain, swelling, loss of function and heat. Many of these signs are difficult to assess objectively. In the neuropathic extremity, pain and disturbance of function may be absent because of neuropathy and thus are poor indicators of inflammation. In addition, swelling and redness are difficult to objectively grade even among experienced clinicians. Most lay people will not be able to understand or accurately evaluate these subtle parameters. Temperature measurements can, however, be easily performed by patients or their spouses and provide quantitative information that has been shown to be predictive of impending ulceration. LAVERY L A, Higgins K R, Lanctot D R, Constantinides G P, Zamorano R G, Armstrong G, Athanasiou K A, Agrawal C M Home monitoring of foot skin temperatures to prevent ulceration Diabetes Care. 2004 November; 27(11):2642, Armstrong D G, et al., Infrared dermal thermometry of the high risk diabetic foot, Physical Therapy, 1997, 77: p. 169-177; Armstrong D G and Lavery L A, Predicting neuropathic ulceration with infrared dermal thermometry, J Amer Podiatr Med Assn, 1997, 87: p. 336-337; Schubert V and Fagrell B, Evaluation of the dynamic cutaneous post-ischaemic hyperaemia and thermal response in elderly subjects and in an area at risk for pressure sores, Clin Physiol, 1991, March; 11(2): p. 169-82; Schubert V, Perbeck L, and Schubert P A, Skin microcirculatory and thermal changes in elderly subjects with early stage of pressure sores, Clin Physiol, 1994, January; 14(1): p. 1-13. Once a patient, such as a diabetic patient, is diagnosed with a “hot spot,” a full ulcer generally develops within a few days to a few weeks of unrecognized repetitive injury. Once a patient identifies an inflamed area, they will require a treatment to reduce the inflammation and protect the injured area from repetitive trauma. The standard treatment for such conditions, that is for a patient who has been diagnosed with the potential of developing an ulcer, is to prescribe an off-loading device. Currently available off-loading devices are not designed to regulate the temperature of the potential ulcer site. The invention is designed to provide immobilization and reduction of pressure and shear to the injured extremity and a mechanism to reduce the local inflammation.

There is a long felt but unmet need for devices and methods for controlling or regulating the temperature of a neuropathic foot, and specifically to regulate the temperature of a potential ulcer site while simultaneously providing the benefits of an off-loading device.

### SUMMARY OF THE INVENTION

In general the present invention provides for a device and methods designed to provide off-loading while simultaneously applying a colder temperature, relative to a target site, to the target site. The target site is typically the location of a potential ulcer such as a “hot spot.”

The present invention specifically provides for a temperature treatment off-loading device that includes an off-loading device, which may be a shoe, and a cooling pack detachably secured to a bottom surface of the off-loading device. The cooling pack may incorporate a rubberized sole. The cooling pack and rubberized sole may be detachably secured to the off-loading device using straps that are insertable into notches in the rubberized sole. An ergonomic insole insert as well as an insole support may be provided within the interior space of the off-loading device.

These and other objects and features of the present invention will be appreciated upon consideration of the following drawings and detailed description.

### BRIEF DESCRIPTION OF THE FIGURES

FIG. 1 illustrates an embodiment of the temperature treatment off-loading device of the present invention.

### DETAILED DESCRIPTION

The present invention regulates the temperature of a target site, which may be potential ulcer site or “hot spot,” while also providing off-loading benefits. The device of the present invention combines an off-loading device with a cool pack. With the use of the present invention, the patient is relieved of the pressure being applied to the target site while the temperature of the target site is simultaneously being controlled with the cool pack. Preferably, the cool pack maintains a temperature of approximately 50° F., and allows the temperature of the target site, which may be elevated, to return to around normal body temperature. As a result, the device eliminates “hot spots” and reduces the potential for an ulcer to form at the target site. In alternative embodiments, cool packs with temperatures other than 50° F. may be used.

By alleviating both pressure and rising temperature at the “hot spot” location prior to the development of an ulcer, a patient's quality of life is increased. Further, the risk of infections developing in the ulcer and the risk of amputation is reduced. The device of the present invention combines an off-loading device with a cool pack to increase a patient's ability to heal and decrease the time period required for rest and off-loading of the injured foot

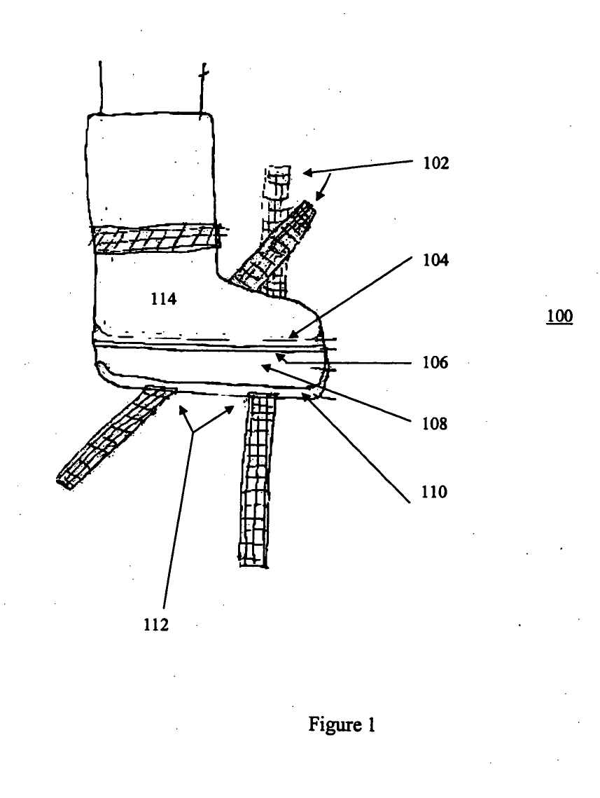

Turning to FIG. 1, a device 100 of the present invention is illustrated. The device 100 includes an off-loading device 114. An ergonomic insole insert 104 is disposed within the interior space of the off-loading device 114, and is preferably positioned to be located at the bottom of a foot when a foot is inserted into the off-loading device 114. An insole support 106 is also present in the interior space of the off-loading device, and is preferably disposed below the ergonomic insole insert 104, i.e., the ergonomic insole insert 104 is placed on top of the insole support 106. The insole support 106 is preferably flexible and displays high thermal conductivity.

A cooling pack 108 is provided. The cooling pack 108 may include alkane, which may be an alkane solution. The cooling pack 108 is detachably secured to the bottom of the off-loading device 114. A rubberized sole 110 is disposed on the bottom of the cooling pack 108. The rubberized sole includes notches 112 through which straps 102 may be inserted. The straps 102 may be a material such as Velcro® that allows for the cooling pack 108 and rubberized sole 110 to be detached and attached to the off-loading device 114. The straps 102 are also usable to secure the device 100 to the foot as well as to secure the cooling pack 108 and rubberized sole 110 to the off-loading device 114. The cooling pack 108 and the rubberized sole 110 are removable so that the cooling pack 108 may be exchanged easily by fastening or undoing the straps 102. In the illustrated embodiment, two straps 102 are provided. More or less than two straps 102 may be incorporated into the device 100.

In an alternative embodiment, rather than being detached to the bottom of the off-loading device, the cool packing may be detached to a side of the off-loading device. In this configuration, the device is utilized to treat the ankle area for fractures, sprains, and other conditions including Charcot's fractures.

Though the invention has been described with respect to specific preferred embodiments, many variations and modifications will become apparent to those skilled in the art. It is therefore intended and expected that the appended claims be interpreted as broadly as possible in view of the prior art in order to include all such variations and modifications.

What is claimed is:

1. A temperature treatment off-loading device, comprising:
an off-loading device;
a cool pack detachably secured to a bottom surface of the off-loading device; and
a rubberized sole coupled to a bottom surface of the cool pack.

2. The temperature treatment off-loading device of claim 1, comprising:
an ergonomic insole insert disposed within an interior space of the off-loading device.

3. The temperature treatment off-loading device of claim 1, comprising:
a notch in the rubberized sole; and
a strap insertable into the notch, wherein the strap is configured to detachably secure the cooling pack and rubberized sole to the off-loading device.

4. The temperature treatment off-loading device of claim 1, comprising:
a plurality of notches in the rubberized sole; and
a plurality of straps insertable into the notches, wherein a single strap is insertable into a single notch and the straps are configured to detachably secure the cooling pack and rubberized sole to the off-loading device.

5. The temperature treatment off-loading device of claim 1, comprising:
an insole support disposed within an interior space of the off-loading device.

6. The temperature treatment off-loading device of claim 5, comprising:
an ergonomic insole insert disposed within an interior space of the off-loading device and on top of the insole support.
